(19) United States
(12) Patent Application Publication
(10) Pub. No.: US 2008/0132508 A1
(43) Pub. Date: Jun. 5, 2008

(54) Method and system for altering dysfunctional lipid metabolism in diabetic complications

(76) Inventors: Mark Kester, Harrisburg, PA (US); Todd E. Fox, Hershey, PA (US)

Correspondence Address: LAURENCE A WEINBERGER, ESQ., 429 EXTON COMMONS, EXTON, PA, 19341, US

(21) Appl. No.: 11/880,446

(22) Filed: Jul. 20, 2007

Related U.S. Application Data
(60) Provisional application No. 60/807,878, filed on Jul. 20, 2006.

(51) Int. Cl. A61K 31/5375; A61P 27/02

(52) U.S. Cl. 514/237.8

(57) ABSTRACT

Diabetic retinopathy is a debilitating complication of diabetes and a leading cause of vision loss, however the fundamental mechanisms contributing to vision loss remain undefined. Several novel observations are described: 1) diabetic retinas demonstrate decreased total ceramide levels; 2) with a concomitant increase in glucosylceramides; 3) which mediates decreased insulin receptor signaling and; 4) cell death in vitro and in vivo. Inhibition of this dysfunctional glycosphingolipid metabolism restores insulin sensitivity. Moreover, elevation in diglycerides and reduction in concentrations of phosphatidic acid or ceramide-1-phosphate also contribute to diabetic complications and insulin resistance. The mechanism responsible for dysfunctional lipid metabolism in diabetic tissues involves reduced caveloin-1-expression within structured membrane microdomains and suggest another target for molecular and pharmacological intervention. Preferred embodiments describe pharmacological and molecular systems and methods to therapeutically alter dysfunctional lipid metabolism and restore selective insulin-dependent kinase cascades as well as membrane integrity.

### GRANT REFERENCE

Work for this invention was funded in part by United States Government, National Institutes of Health Grant # 1RO1 EY015800-02. The Government may have certain rights in this invention.

### BACKGROUND OF THE INVENTION

Diabetes has many long term complications, including nephropathy, neuropathy and retinopathy. Retinopathy is primarily a vascular disease brought on by high glucose and resulting damage to vascular tissue with subsequent damage to retinal tissues. Retinal degeneration due to neuronal cell death is an underlying cause of many visual diseases including retinitis pigmentosa, macular degeneration and diabetic retinopathy. Current treatment options for retinopathy include surgery and laser treatment, neither of which is completely successful in preventing blindness. For these and other reasons, there is a need for the present invention.

### DETAILED DESCRIPTION OF THE PREFERRED EMBODIMENT

The methods by which the objects, features and advantages of the present invention are achieved will now be described in more detail. These particulars provide a more precise description of the invention for the purpose of enabling one of ordinary skill in the art to practice the invention, but without limiting the invention to the specific embodiments described.

### DEFINITIONS

As used herein and in the appended claims, the singular forms “a”, “an” and “the” include plural references unless the context clearly dictates otherwise.

The term “co-administration” or “co-administering” as used herein refer to the administration of a compound before, concurrently, or after the administration of another compound.

As used herein, “therapeutically effective amount” refers to an amount, which is effective in reducing, eliminating, treating, preventing or controlling the symptoms of the herein-described diseases, disorders, or conditions, for example, a therapeutically effective amount of a compound will slow the onset of diabetic retinopathy or prevent or decrease apoptosis of cells associated with diabetic retinopathy.

As used herein, unless otherwise defined in conjunction with specific diseases or disorders, the term “treating” refers to: (i) preventing a disease, disorder or condition from occurring in an mammal or human that may be predisposed to the disease, disorder and/or condition but has not yet been diagnosed as having it; (ii) inhibiting the disease, disorder or condition, i.e., arresting its development; and/or (iii) relieving the disease, disorder or condition, i.e., causing regression of the disease, disorder and/or condition.

While the present inventors are using diabetic retinopathy as a model to prevent the death of retinal neurons, the present inventors contemplate that the methods and compositions of the invention may be used to treat other conditions associated with or resulting from diabetic complications, including without limitation, neuropathy, nephropathy, cardiomyopathy, microangiopathy, and macroangiopathy, insulin resistance, cataracts, hyperglycemia, hypercholesterolemia, hypertension, hyperinsulinemia, hyperlipidemia, coronary diseases, stroke, heart attack, and peripheral arterial disease, atherosclerotic lesions, and tissue ischemia, including myocardial ischemia, death of vasculature cells, death of pancreatic islet cells, obesity, all metabolic stress-induced diabetic complications that affect vasculature (vascular disease), for example, ischemic vasculature, coronary and vascular hyperplasia and hypertrophy, and muscular hypertrophy.

As used herein, the term “mammal” includes but is not limited to a horse, cat, dog, rat, mouse, cow, pig or human.

### Glucosylceramide (GleCer)

The present inventors demonstrate for the first time that increased glycosphingolipid synthesis through glucosylceramides may contribute to cell death in diabetic retinopathy.

Without wishing to be bound by this theory, the present inventors contemplate that inhibition of glycosphingolipid metabolism increases insulin sensitivity in retinal neurons and/or other cell types in the retina, including for example, microglia, macroglia, vacular endothelial cells, and periocytes. The present inventors contemplate that compounds that inhibit the formation of glycosphingolipids, for example, by decreasing glucosylceramide synthase levels or by inhibiting glucosylceramide synthase may used in the present invention. Compounds, suitable for use with the invention include, but are not limited to, a drug, a prodrug, a small molecule, an imino sugar, a linear or cyclic peptide, a protein, a carbohydrate or oligosaccaride, or a nucleic acid such as a DNA or RNA oligonucleotide, plasmid DNA, siRNA, ribozyme, or an inhibitor.

The present inventors contemplate a method of treating diabetic complications which comprises administering to a mammal in need of treatment a therapeutically effective amount of a compound that inhibits glycosphingolipid metabolism. In one embodiment, the diabetic complication is diabetic retinopathy.

In another embodiment, the present inventors contemplate that the methods of the invention can also be used in the treatment of other diseases or conditions of retinal inflammation and neurodegeneration, for example, Farber's disease (acid ceramidase), Tay-Sachs/Sandhoff (hexosaminidase A or B), Gaucher's (glucosylceramidase), Krabbe's (galactoslyceramidase) and Niemann Pick disease (sphingomyelinase). In another embodiment, the present inventors contemplate that the methods of the invention can also be used in the treatment of other diseases or conditions of glucosylceramide synthesis-related diseases and disorders, such as diabetic retinopathy. In another embodiment, the present inventors contemplate that the methods of the invention can also be used in the treatment of other conditions or diseases associated with neuronal apoptosis such as diabetic retinopathy. In one embodiment, the methods of the present invention can be used in the treatment of diabetic retinopathy or other diabetic complications.

In one embodiment of the present invention, a method for treating diabetic complications in mammals comprises administering to a mammal in need of such treatment a therapeutically effective amount of a compound that inhibits or decreases glycosphingolipid synthesis. In one embodiment of the present invention, a method for treating diabetic retinopathy in mammals comprising administering to a mammal in need of such treatment a therapeutically effective amount of a compound that inhibits or decreases glycosphingolipid synthesis is provided. In another aspect of the invention, the compound is an inhibitor of glucosylceramide synthase. In another aspect of the invention, the inhibitor includes PPMP, PDMP, or NB-DGJ or any derivative or analog thereof or any derivative of ceramide that inhibits glycosphingolipid metabolism, in particular, glucosylceramide synthase, for use in the present invention. In another aspect of the invention, the compound is a recombinant glucosylceramidase, for example, cerezyme.

In another embodiment of the invention, a method for treating diabetic retinopathy comprising administering to a mammal a therapeutically effective amount of a compound that decreases levels of glucosylceramide is provided. In another embodiment of the methods of the invention, a compound that inhibits glucosylceramide synthase is administered. In another aspect of the invention, the compound is an inhibitor of glucosylceramide synthase. In one embodiment, the glucosylceramide synthesis inhibitor is an imino sugar. In another aspect of the embodiments, the imide sugar is N-butyldeoxynojirimycin, N-butyldeoxygalactonojirimycin (NB-DGJ), or N-nonyldeoxynojirimycin. In another embodiment, the inhibitor of glucosylceramide synthesis is 1-phenyl-2-decanoylamino-3-morpholino-1-propanol (DMP), D-threo-1-phenyl-2-decanoylamino-3-morpholino-1-propanol and structurally related analogues thereof. In another embodiment, the inhibitor of glucosylceramide synthesis is 1-phenyl-2-palmitoyl-amino-3-morpholino-1-propanol (PPMP) and structurally related analogues thereof.

The present inventors also contemplate that a compound or a combination of compounds capable of decreasing glucosylceramide synthase levels or inhibiting glucosylceramide synthase may be administered to a mammal in need thereof. In another aspect, the compound is administered topically.

In another embodiment, the method comprises administering at least one compound capable of decreasing glucosylceramide synthase levels or inhibiting glucosylceramide synthase and decreasing DAG levels or increasing PA levels, useful for simultaneous, sequential or separate treatment in the treatment of a diabetic retinopathy or other conditions or diseases, including without limitation Farber's disease (acid ceramidase), Tay-Sachs/Sandhoff (hexosaminidase A or B), Gaucher's (glucosylceramidase), Krabbe's (galactoslyceramidase), Niemann Pick disease (sphingomyelinase), diseases or conditions of glucosylceramide synthesis-related diseases and disorders, and diseases or conditions associated with neuronal apoptosis such as diabetic retinopathy.

The change in gluycosylceramide synthesis due to the administration of such compounds can be readily detected, e.g., by obtaining a biopsy sample, or by assaying in vitro the levels of activities of enzymes involved in glucosylceramide synthesis, or the levels of mRNAs encoding such enzymes, or any combination of the foregoing. Such assays can be performed before and after the administration of the compound.

In one embodiment, a compound decreasing glucosylceramide synthase levels and/or inhibiting glucosylceramide synthase activity is administered. In another embodiment, a compound capable of increasing the rate of neuronal glycolipid degradation, e.g., a glucosylceramide glucosidase, is administered. Any suitable methods for administering a compound available in the art can be used according to the present invention.

Various delivery systems are known and can be used to administer a compound or pharmaceutical composition for use in the methods of the present invention, e.g., encapsulation in liposomes (see, e.g., Kester et al, patent application Ser. No. 10/835,520, Method and System for Systemic Delivery of Growth Arresting, Lipid-derived Bioactive Compounds), microparticles, microcapsules, recombinant cells capable of expressing the compound, receptor-mediated endocytosis, construction of a nucleic acid as part of a retroviral or other vector, and the like. Methods of introduction can be enteral or parenteral and include but are not limited to intradermal, intramuscular, intrapertoneal, intravenous, intraocular, subcutaneous, intranasal, epidural, and oral routes. The compounds may be administered by any route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

### Compositions

The present invention also provides pharmaceutical compositions for use in treating diabetic complications, including diabetic retinopathy. Such compositions may comprise a therapeutically effective amount of a compound capable of decreasing glucosylceramide synthase levels or inhibiting glucosylceramide synthase and a pharmaceutically acceptable carrier.

The present invention also provides pharmaceutical compositions. Such compositions comprise a therapeutically effective amount of a compound capable of decreasing glucosylceramide synthase levels or inhibiting glucosylceramide synthase and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical Sciences” by E. W. Martin. A composition will contain a therapeutically effective amount of a compound capable of decreasing glucosylceramide synthase levels or inhibiting glucosylceramide synthase, together with a suitable amount of carrier so as to provide the form for proper administration to the subject. The formulation should suit the mode of administration.

The route of administration may be any route which effectively transfers the compound to the appropriate or desired site of action. In one aspect, the compound may be formulated in any suitable ophthalmic formulation such as solutions, suspensions, ointments, and the like. The formulations can include other components known to those skilled in the art of formulating ophthalmic products. For example, the formulations may include ophthalmically acceptable preservatives, surfactants, viscosity enhancers, penetration enhancers, and buffers. The formulations may be applied topically to the eye of a mammal in need of treatment thereof according to the routine discretion of a skilled clinician. In another aspect, the compositions of the present invention are administered through injectable compositions. Among the possible injection modes suitable for administration, the compositions can be administered through subconjunctival, subtenonia, episcleral, subcutaneous, intravenous or intravitreous injections.

The amount of the compound capable of decreasing glucosylceramide synthase levels or inhibiting glucosylceramide synthase which will be effective in the treatment of diabetic retinopathy and related disorders can be determined by standard clinical techniques based on the present description. In addition, in vitro assays and in vivo models, for example, streptozotocin (stz)-diabetes in rats and ins-2 akita mice, may optionally be employed to help identify optimal dosage ranges. The precise dose to be employed in the formulation will also depend on the route of administration, and the seriousness of the disease or disorder, and should be decided according to the judgment of the practitioner and each subject's circumstances. Effective doses may be extrapolated from dose-response curves derived from in vitro or animal model test systems.

### DAG

Diacylglycerol (DAG) plays a central role in both the synthesis of complex lipids and in intracellular signaling. The present inventors contemplate that DAG plays a key role in the death of retinal neurons, in particular in diabetic retinopathy. Diacylglycerol, a second messenger, activates other molecules within the cell including protein kinase C (PKC) which is central to numerous biological processes, including the regulation of cell growth and differentiation and importantly the deactivation of the insulin receptor.

Without wishing to be bound by this theory, the present inventors contemplate that DAG plays a dual role in the treatment of diabetic retinopathy. First, it is hypothesized that decreased levels of DAG will result in decreased PKC activity. Lessening the amount of DAG available to activate protein kinase C (PKC) results in less deactivation of the insulin receptor and increases a cell's insulin sensitivity. Second, decreasing levels of DAG by converting DAG into PA through phosphorylation activates the mammalian target of rapamycin (mTOR) signaling pathway of which p70 S6K, a downstream effector of mTOR, is believed to promote insulin-induced survival of retinal neurons.

The present inventors contemplate that a compound that increases PA levels and/or decreases DAG levels may used in the present invention. Compounds, suitable for use with the invention include, but are not limited to, a drug, a prodrug, a small molecule, an imino sugar, a linear or cyclic peptide, a protein, a carbohydrate or oligosaccaride, or a nucleic acid such as a DNA or RNA oligonucleotide, plasmid DNA, siRNA, ribozyme, or an inhibitor.

In one embodiment of the present invention, a method for treating diabetic complications in mammals comprising administering to a mammal in need of such treatment a therapeutically effective amount of a compound that increases intracellular levels of PA is provided. In another embodiment, the diabetic complication is diabetic retinopathy.

In another aspect, the present invention contemplates a method of increasing intracellular levels of PA. In another aspect, levels of PA are increased by converting DAG into PA, by administration of exogenous PA, by administration of nondegradable PA or short chain PA having 10 carbons or less, or by activation of phospholipase D (PLD). In one aspect, the short chain PA have 10 carbons or less in the in the sn-1 and/or sn-2 position of the lipid. In another aspect, the short chain PA have 10 carbons or less in the in the sn-1 and/or sn-2 position of the lipid and have from n=2 to n=10 saturated carbon bonds as compared to more physiological species having n=16 or longer carbon units. In another aspect, short chain PA are cell-permeable.

The change in PA or DAG levels due to the administration of such compounds can be readily detected, e.g., by obtaining a biopsy sample, or by assaying in vitro the levels of activities of enzymes involved in PA or DAG synthesis, or the levels of mRNAs encoding such enzymes, or any combination of the foregoing. Such assays can be performed before and after the administration of the compound.

Thus, the present invention includes a method of increasing PA concentrations, thereby restoring mTOR signaling, as a therapeutic modality to reduce inflammatory cytokine-induced neuronal cell death in diabetic retinopathy. Inflammatory cytokines include but are not limited to interleukin1Beta (IL-1β), interleukin 6 (IL-6), tumor necrosis factor alpha (TNFα).

In another embodiment of the invention, a method of treating diabetic retinopathy comprises regulating cell survival by decreasing the amount of DAG available to activate PKC. In another aspect of the invention, the method includes decreasing the amount of intracellular DAG by converting DAG to phosphatidic acid (PA) using various enzymes which catalyze the conversion of DAG to PA. In one aspect, DAG is converted to PA using a DAG kinase.

Levels of DAG and/or PA can be detected using a number of methods known to one skilled in the art, including but not limited to Northern and Western blots, PCR, and a DAG kinase assay. The change in PA, DAG, or various enzymes which catalyze the conversion of DAG to PA levels or the change in the activity of various enzymes which catalyze the conversion of DAG to PA due to the administration of such compounds can be readily detected, e.g., by obtaining a biopsy sample, or by assaying in vitro the levels of activities of enzymes involved in PA or DAG synthesis, or the levels of mRNAs encoding such enzymes, or any combination of the foregoing. Such assays may be performed before and after the administration of the compound.

In one embodiment, the methods of the present invention can be used in the treatment of diabetic retinopathy or other diabetic complications. In another embodiment, the present inventors contemplate that the methods of the invention can also be used in the treatment of other diseases or conditions of retinal inflammation and neurodegeneration, for example, Farber's disease (acid ceramidase), Tay-Sachs/Sandhoff (hexosaminidase A or B), Gaucher's (glucosylceramidase), Krabbe's (galactoslyceramidase) and Niemann Pick disease (sphingomyelinase). In another embodiment, the present inventors contemplate that the methods of the invention can also be used in the treatment of other diseases or conditions of glucosylceramide synthesis-related diseases and disorders, such as diabetic retinopathy or diseases or conditions associated with neuronal apoptosis.

In one embodiment of the present invention, a method for treating diabetic retinopathy in mammals comprising administering to a mammal in need of such treatment a therapeutically effective amount of a compound that increases PA levels is provided. In another aspect of the invention, the compound is a diacylglycerol kinase (DGK). In another aspect of the invention, the compound activates phospholipase D (PLD).

In another embodiment of the invention, a method for treating diabetic retinopathy comprises administering to a mammal a therapeutically effective amount of a compound that increases levels of PA. In another aspect of the invention, a method for treating diabetic retinopathy by administering to a mammal a compound that activates mTOR or a downstream effector of mTOR is provided. In another embodiment of the invention, a method for treating diabetic retinopathy comprises administering to a mammal a compound that decreases DAG levels. In another aspect of the invention, a method for treating diabetic retinopathy comprises administering to a mammal a compound that decreases PKC activity or a downstream effector of PKC.

The change in mTOR or PKC levels due to the administration of such compounds can be readily detected, e.g., by obtaining a biopsy sample, or by assaying in vitro the levels of activities of enzymes involved in mTOR or PKC synthesis or signaling pathways, or the levels of mRNAs encoding such enzymes, or any combination of the foregoing. Such assays can be performed before and after the administration of the compound

The present inventors also contemplate that a compound or a combination of compounds capable of increasing PA levels and/or decreasing DAG may be administered or co-administered to a mammal in need thereof. In another aspect, the compound is administered topically.

In another embodiment, the method comprises administering at least one compound capable of decreasing glucosylceramide synthase levels or inhibiting glucosylceramide synthase and at least one compound capable of decreasing DAG levels or increasing PA levels, useful for simultaneous, sequential or separate treatment in the treatment of diabetic retinopathy or other conditions or diseases associated with neuronal apoptosis or diseases or conditions of retinal inflammation and neurodegeneration, for example, Farber's disease (acid ceramidase), Tay-Sachs/Sandhoff (hexosaminidase A or B), Gaucher's (glucosylceramidase), Krabbe's (galactoslyceramidase) and Niemann Pick disease (sphingomyelinase). In another embodiment, the present inventors contemplate that the methods of the invention can also be used in the treatment of other diseases or conditions of glucosylceramide synthesis-related diseases and disorders, such as diabetic retinopathy.

Any suitable methods for administering a compound available in the art can be used according to the present invention. Various delivery systems are known and can be used to administer a compound or pharmaceutical composition for use in the methods of the present invention, e.g., encapsulation in liposomes (see, e.g., Kester et al, patent application Ser. No. 10/835,520, Method and System for Systemic Delivery of Growth Arresting, Lipid-derived Bioactive Compounds), microparticles, microcapsules, recombinant cells capable of expressing the compound, receptor-mediated endocytosis, construction of a nucleic acid as part of a retroviral or other vector, and the like. Methods of introduction can be enteral or parenteral and include but are not limited to intradermal, intramuscular, intraocular, intraperitoneal, intravenous, subcutaneous, intranasal, epidural, and oral routes. The compounds may be administered by any route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

### Compositions

The present invention also provides pharmaceutical compositions for use in treating diabetic complications, including diabetic retinopathy. Such compositions may comprise a therapeutically effective amount of a compound capable of decreasing DAG levels and/or increasing PA levels and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical Sciences” by E. W. Martin. Such compositions will contain a therapeutically effective amount of a compound capable of decreasing DAG levels and/or increasing PA levels, preferably in purified form, together with a suitable amount of carrier so as to provide the form for proper administration to the subject. The formulation should suit the mode of administration.

The route of administration may be any route which effectively transfers the compound to the appropriate or desired site of action. The compositions of the present invention may be administered by any means suitable for the condition to be treated, which may depend on the need for site-specific treatment or quantity of drug to be delivered. Any appropriate route of administration may be employed, for example, parenteral, intravenous, subcutaneous, intramuscular, intracranial, intraorbital ophthalmic, intraventricular, intracapsular, intraspinal, intracisternal, intraperitoneal, intranasal, aerosol, or oral administration. Methods well known in the art for making formulations are found in, for example, “Remington's Pharmaceutical Sciences.”

The compound can be formulated in any suitable ophthalmic formulation such as solutions, suspensions, ointments, and the like. The formulations can include other components known to those skilled in the art of formulating ophthalmically products. For example, the formulations can include ophthalmically acceptable preservatives, surfactants, viscosity enhancers, penetration enhancers, and buffers. The formulations are applied topically to the eye of a mammal in need of treatment thereof according to the routine discretion of a skilled clinician.

The amount of the compound capable of decreasing DAG levels and/or increasing PA levels of the invention which will be effective in the treatment of diabetic retinopathy and related disorders can be determined by standard clinical techniques based on the present description. In addition, in vitro assays and in vivo models, for example, streptozotocin (stz)-diabetes in rats and ins-2 akita mice, may optionally be employed to help identify optimal dosage ranges. The precise dose to be employed in the formulation will also depend on the route of administration, and the seriousness of the disease or disorder, and should be decided according to the judgment of the practitioner and each subject's circumstances. Effective doses may be extrapolated from dose-response curves derived from in vitro or animal model test systems.

### EXAMPLES

Examples are provided in Appendices A and B, which are incorporated into this Specification in its entirety.

Although various aspects of the composition are described in detail, it will be apparent to one skilled in the art that modifications, substitutions, and additions may be made without departing from the spirit and scope of the invention. All patents, patent applications, articles and publications mentioned herein, both supra and infra, are hereby incorporated herein by reference.

What is claimed is:

1. A method for treating diabetic retinopathy in mammals comprising:
administering to a mammal in need of such treatment a therapeutically effective amount of a compound that inhibits glycosphingolipid synthesis.

2. The method of claim 1, wherein the inhibitor is an inhibitor of glucosylceramide synthase.

3. The method of claim 2, wherein the inhibitor is PPMP, PDMP or NB-DGJ.

4. A method for treating neuronal apoptosis associated with diabetic retinopathy comprising:
administering to a mammal an effective amount of a compound that decreases levels of glucosylceramide in retinal neuronal cells.

5. The method of claim 4 wherein the level of glucosylceramide is decreased using an inhibitor of glucosylceramide synthase.

6. The method of claim 5 wherein the compound is an inhibitor of glucosylceramide synthase is PPMP, PDMP or NB-DGJ.

7. The method of claim 4, wherein the compound is administered topically.

8. The method of claim 4, further comprising:
administering a compound that increases levels of phosphatidic acid (PA).

9. The method of claim 4, further comprising:
administering a compound that decreases levels of diacylglycerol (DAG).

10. A pharmaceutical composition useful for treating diabetic retinopathy comprising:
a compound that inhibits glycosphingolipid synthesis and a pharmaceutically acceptable carrier.

11. The composition of claim 10 wherein the compound is an inhibitor of glucosylceramide synthase selected from PPMP, PDMP or NB-DGJ.

12. The composition of claim 10 further comprising:
a compound that increases PA levels.

13. The composition of claim 10 further comprising:
a compound that decreases DAG levels.

14. A method for treating diabetic retinopathy in mammals comprising:
administering to a mammal in need of such treatment a therapeutically effective amount of a compound that increases levels of PA in retinal cells.

15. The method of claim 14 wherein said compound increases levels of PA by phosphorylating DAG into PA.

16. The method of claim 14 wherein said compound increase levels of PA by activating phospholipase D (PLD).

17. The method of claim 14, further comprising:
administering a compound that decreases levels of DAG.

18. The method of claim 14, further comprising:
administering a compound that inhibits glycosphingolipid synthesis.

19. The method of claim 18 wherein the inhibitor is an inhibitor of glucosylceramide synthase.

20. The method of claim 19, further comprising wherein the inhibitor is PPMP, PDMP or NB-DGJ.

21. A method for treating neuronal apoptosis associated with diabetic retinopathy comprising:
administering to a mammal an effective amount of a compound that increases levels of PA in retinal neuronal cells.

22. The method of claim 21 wherein said compound increases levels of PA by phosphorylating DAG into PA.

23. The method of claim 21 wherein said compound increases levels of PA by activating phospholipase D (PLD).

24. The method of claim 21, further comprising:
administering a compound that decreases levels of DAG.

25. The method of claim 21, further comprising:
administering a compound that inhibits glycosphingolipid synthesis.

26. The method of claim 25 wherein the compound is an inhibitor of glucosylceramide synthase.

27. The method of claim 26, wherein the inhibitor is PPMP, PDMP or NB-DGJ.

28. The method of claim 21, wherein the compound is administered topically.

29. A pharmaceutical composition useful for treating diabetic retinopathy comprising:
a compound that increases PA levels and a pharmaceutically acceptable carrier.

30. The pharmaceutical composition of claim 29 further comprising:
a compound that decreases DAG levels.

31. The pharmaceutical composition of claim 29, wherein said compound increases levels of PA by phosphorylating DAG into PA.

32. The pharmaceutical composition of claim 29, further comprising:
wherein said compound increases levels of PA by activating phospholipase D (PLD)

33. The pharmaceutical composition of claim 29 further comprising:
a compound that inhibits glycosphingolipid synthesis.

34. The pharmaceutical composition of claim 33 wherein the compound is an inhibitor of glucosylceramide synthase selected from PPMP, PDMP or NB-DGJ.

35. The method of claim 4, wherein the reduction of glycosphingolipid accumulation is a recombinant form of an enzyme that degrades glycosphingolipids

36. The method of claim 5, wherein the recombinant enzyme is glucosylcerebrosidase or glucosylceramidase.

37. A method for treating diabetic retinopathy in mammals comprising: administration to a mammal in need of such a treatment a therapeutically effective amount of compound that elevates caveolin-1 expression levels, leads to reduction of glucosylceramide accumulation.

38. The method of claim 4, further comprising administering a compound that increases levels of ceramide-1-phosphate
